(19) United States
(12) Patent Application Publication
(10) Pub. No.: US 2006/0088612 A1
(43) Pub. Date: Apr. 27, 2006

(54) Composition for treatment of topical infection and methods of making and using same

(76) Inventors: Hannah Naomi Sivak, Gilbert, AZ (US); Alberto Alvaro Iglesias, Rosario (AR); Miguel Angel Ballicora, Skokie, IL (US)

Correspondence Address: SNELL & WILMER;ONE ARIZONA CENTER, 400 EAST VAN BUREN, PHOENIX, AZ, 850040001, US

(21) Appl. No.: 11/218,872

(22) Filed: Sep. 2, 2005

Related U.S. Application Data
(60) Provisional application No. 60/606,679, filed on Sep. 2, 2004.

(51) Int. Cl. A61K 36/53; A61K 36/61

(52) U.S. Cl. 424/745; 424/769

(57) ABSTRACT

Compositions and methods for the prevention and treatment of topical infections using essential oil or extract from Minthostachys mollis (Peperina) or other herbs of the family Lamiaceae, or any combination of its individual chemical components and an acceptable carrier are disclosed.

### CROSS-REFERENCE TO RELATED APPLICATIONS

This application claims the benefit of U.S. Provisional Application No. 60/606,679, filed on Sep. 2, 2004.

### FIELD OF INVENTION

The present invention generally relates to compositions and methods for treating topical infections and more particularly to compositions including an extract from an herb from the family Lamiaceae and to methods of using and forming the composition.

### BACKGROUND OF THE INVENTION

Topical infections, such as bacterial, viral, or fungal infections often inhibit wound healing. Such infections can lead to additional health problems such as sepsis or the like and are therefore often treated with antimicrobial agents.

One particular viral infection, infection by Herpes simplex virus (HSV), can affect the oral or perioral region, eyes, nongenital and genital skin or mucous membrane. Symptoms of HSV infections can appear suddenly and include appearance of multiple, small, grouped blisters bordered by an inflammatory, erythematous base. Recurrent HSV infections of the lips and perioral area are estimated to occur in about 20 to 40 percent of the population worldwide

HSV is a DNA virus; like all viruses, it needs the host cell to multiply. The scientific name of the virus is Human herpesvirus, Genus Simplexvirus, Subfamily Herpesviridae, Family Alphaherpesvirinae, host Homo sapiens.

Whatever the primary site of an HSV infection, the ability of HSV to enter, undergo latency, and survive in neural ganglia shields the virus from elimination by immune responses and ensures that a person infected with the virus will endure recurring symptomatic events during his/her life. When left untreated, the symptoms generally subside in 1 to 2 weeks.

Recurrent HSV infection is usually preceded by pain, burning, tingling, and itching. Precipitating factors for HSV recurrence may include exposure to sunlight, fever, trauma to the area of primary infection, trigeminal nerve manipulation (in the case of oral infection), menstruation, and emotional stress. Recurrences may occur as frequently as once per month or as infrequently as twice per year. Patients usually develop lesions at similar sites with each recurrence, although recurrences can occur at more distant sites.

Antiviral medications given orally like the prescription antiviral acyclovir have been used to treat HSV. Such treatments are generally undesirable because the active ingredient must travel a circuitous rout to the infected area and must travel through the digestive tract, which may cause degredation of the active ingredient.

Topical medications for HSV also exist. Such medications include ointments containing non-rescription ingredients such as menthol (anti-itch), camphor (analgesic), phenol (anesthetic), allantoin (anti-inflammatory), docosanol (behenyl alcohol), or prescription medications such as penciclovir and acyclovir creams. While such medications may be effective at alleviating some symptoms of HSV, such medications, such as docosanol have been found to be no more effective than a placebo and others cause irritation and/or pain near the infected area. Accordingly, improved compositions and methods for treating HSV and other infections are desired.

Peperina is an aromatic plant that grows wild in the valleys of the province of Cordoba, in Argentina. It has been used for its medicinal properties for centuries. The botanical name of Peperina is Minthostachys mollis but other names, such as Minthostachys verticillata, ystropogon mollis Kth., have been used to describe the same substance.

Peperina is a member of the “mint-like” aromatic herbs (of the genus Minthostachys and the Family Lamiaceae), which are limited to mountain zones of South America, with about 12 species distributed at various altitudes from Venezuela to Argentina. Pollen morphology has indicated that the genus is most closely linked to Pycnanthemum and Bystropogon of the Canary Islands. Common names in other regions of South America for this and other species of the same genus include muña, tipo and poleo.

It has been shown that the essential oil of Peperina has antiviral activity in vitro against herpes simplex type 1 (HSV-1). The oil also has activity against bacteria and fungi and the pseudorabies virus. However, it is believed that direct application of Peperina to the skin or a mucosal area will irritate the area, which may cause pain and further damage to the area, and would require frequent application of the oil to have an efficacious effect for the treatment of the infection. Accordingly, compositions including herbs from the family Lamiaceae, and in particular, Peperina essential oil, which do not irritate the infected area and which are configured to reside proximate the infected area for an extended period of time are desired.

### SUMMARY OF THE INVENTION

The present invention provides a composition including an extract from an herb from the family Lamiaceae for the treatment of topical infections. While the ways in which the invention addresses the various drawbacks of known compositions and methods of treating infections will be described in more detail below, in general, the present invention provides a composition including a relatively long-lasting herbal antimicrobial agent and a carrier configured to maintain the composition in contact with an infected area for a prolonged period of time.

In accordance with one embodiment of the invention, a composition includes a carrier including an agent to facilitate extended contact between the composition and an infected area and an effective amount of an extract from an herb from the family Lamiaceae (e.g., Peperina essential oil). In accordance with various aspects of this embodiment the composition includes about 0.1 to about 2 weight percent Peperina essential oil and about 5 to about 10 weight percent of a thickening agent such as siliconized bee wax or honey.

In accordance with another embodiment of the invention, a composition includes a carrier including an ingredient to facilitate tissue repair and an effective amount of an extract from an herb from the family Lamiaceae (e.g., Peperina essential oil). In accordance with various aspects of this embodiment, the composition includes about 0.1 to about 2 weight percent Peperina essential oil and about 2 to about 4 percent of an essential fatty acid such as linolenic or linoleic acid.

In accordance with a further embodiment of the invention, a composition includes a carrier including an emollient and/or moisturizing agent to mitigate irritation of the infected area and an effective amount of an extract from an herb from the family Lamiaceae (e.g., Peperina essential oil). In accordance with various aspects of this embodiment, the composition includes about 0.1 to about 2 weight percent Peperina essential oil and about 10 to about 20 weight percent of a moisturizing agent such as lanolin.

In accordance with yet a further embodiment of the invention, a composition includes a carrier including an antimicrobial agent and an effective amount of an extract from an herb from the family Lamiaceae (e.g., Peperina essential oil). In accordance with various aspects of this embodiment, the composition includes about 0.1 to about 2 weight percent Peperina essential oil and about 0.01 to about 0.2 weight percent of a antimicrobial agent such as bee propolis extract.

In accordance with yet another embodiment of the invention, a composition is formed by heating the solid ingredients until they are softened enough to mix.

In accordance with yet a further embodiment of the invention, a method of using a composition includes applying a composition including of an extract from an herb from the family Lamiaceae (e.g., Peperina essential oil) to an infected area on an animal. In accordance with various aspects of this embodiment, the composition includes about 1-10 mg of Peperina essential oil per dose depending on the area of the lesion. In accordance with another aspect of this embodiment, the composition includes a thickening agent. In accordance with yet another aspect, the composition includes a moisturizing agent. In accordance with a further aspect of this exemplary embodiment, the composition includes a repairing agent.

### DETAILED DESCRIPTION

The present invention generally relates to compositions and methods of managing one or more conditions in a patient having a topical infection. Although the composition and methods described herein may be used to treat a variety of topical infections, specific embodiments are described below in connection with the treatment of a Herpes simplex virus (HSV). Those skilled in the art will appreciate that the compositions of the present invention are not limited to the treatment of HSV and that the compositions may be used for the treatment of bacterial, fungal, and similar infections.

In the case of HSV, the composition is topically applied to a patient who exhibits herpetic skin or labial or mucous membrane or ocular lesions. The composition, which includes a therapeutically effective amount of an extract from an herb from the family Lamiaceae (e.g., Peperina essential oil) in a pharmaceutically acceptable carrier, is applied on or proximate the lesions until the appearance of the lesions diminishes or vanishes. As set forth in more detail below, the composition may be include one or more additional pharmaceutical compositions to facilitate managing and healing of the infection.

As used herein, the term “Peperina essential oil” includes essential oils obtained from the genus Myntostachis, from dried or fresh aerial plant material harvested from the wild, or cultivated and obtained by steam distillation, by extraction with supercritical carbon dioxide, or any method used to extract the volatile components of the plant. Compositions of Peperina essential oil, like that of other plants, may vary greatly and depend on the growth conditions, time of collection of the plant material, etc. For example, Peperina essential oil collected in the province of Cordoba, Argentina, contains about 8.9-58.3% of pulegone, about 24.1-73.3 of menthone, and includes other components such as limonene, isomenthone, piperitone, trans-ocimene and terpinene-4-ol; but the sum of menthone plus pulegone is generally constant and around 80%. Composition of essential oils obtained from plant material collected from other provinces (with different ecological conditions) varied even further, and in some cases thymol, carvacrol and/or carvone were present.

An amount of herb extract (e.g., Peperina essential oil) necessary to bring about prevention and/or therapeutic treatment of an infection may vary according to application, the source of the oil, the method employed to extract the essential oil, the amount and type of any additional ingredients used, particularly those that appear to exhibit synergistic effects, the skin type of the user, and, where present, the severity and extent of skin damage. Generally, the Peperina essential oil composition is topically applied in effective amounts to skin or mucosa areas which are affected, or have been affected, by the virus. In accordance with various exemplary embodiments of the invention, the composition contains from about 0.1 to about 10 weight percent, preferably from about 0.1 to about 1 weight percent Peperina essential oil, and more preferably from about 0.2 to about 1 weight percent.

The Peperina essential oil is applied in admixture with an acceptable carrier (e.g., a lotion, cream, ointment, serum, liquid drop, sunscreen, or the like). Sunscreen, balms, and lotions may be particularly desirable carriers since sunscreens mitigate exposure to UVA and UVB rays that may bring about herpes virus recurrence, creams are often used to treat sunburn or burns produced by therapeutical radiation used to treat cancer, and balms are often used after laser treatment and application of peels. In general, the carrier is configured to facilitate topical application and provide additional therapeutic effects such as moisturizing the affected skin areas.

The carrier of the present invention is configured to facilitate topical application, and optionally form a film or layer on a surface (e.g., skin) to which it is applied so as to localize the active ingredient and maintain the active ingredient in contact with the infected area for an extended period of time. Exemplary carriers suitable for the present invention include one or more of the following: emollients such as natural oils and waxes, silicone oils, hyaluronic acid, glyceride derivatives, fatty acids or fatty acid esters or alcohols or alcohol ethers, lanolin and derivatives, polyhydric alcohols or esters, wax esters, sterols, phospholipids and the like, emulsifiers (nonionic, cationic or anionic)--some of the emollients inherently possess emulsifying properties. These general ingredients can be formulated into, for example, a cream, a lotion, a gel, a lip balm, a gel, or into solid sticks by utilization of different proportions of the ingredients and/or by inclusion of thickening agents such as gums or other forms of hydrophilic colloids.

In accordance with various exemplary embodiments of the invention, the carrier includes a thickening agent to facilitate maintaining the composition in contact with an infected area for an extended period of time. Exemplary thickening include siliconized bee wax, honey, and other waxy ingredients used in the industry for this purpose. In accordance with various aspects of this embodiment of the invention, the composition includes about 5 to about 10 weight percent of a thickening agent.

In accordance with additional embodiments of the invention, the carrier includes emollients and/or moisturizers such as lanolin, glycol ester (e.g., Syncrowax ERL-C), castor oil, jojoba oil, safflower oil, evening promrose oil. In accordance with one particular aspect of this embodiment, a composition includes about 10 to about 20 weight percent lanolin, about 5 to about 10 weight percent glycol ester, about 30 to about 60 weight percent castor oil, about 20 to about 40 weight percent jojoba oil, about 2 to about 4 weight percent safflower oil, and about 2 to about 4 weight percent evening of primrose oil.

The carrier may also include occlusive agents such as Syncrowax HCL-C and/or triehenin, which are present in an amount of about 0 to about 30 weight percent of the composition. By way of particular example, a carrier in accordance with an exemplary embodiment of the invention includes about 5 to about 20 weight percent Syncrowax HGL-C and about 5 to about 10 weight percent of a skin conditioner such as tribehenin.

Carriers in accordance with various additional exemplary embodiments of the invention include at least one other active ingredient in addition to the Peperina essential oil. Such additional active ingredients are compatible with essential oils.

The additional active ingredient may be antiviral, antibacterial and/or antifungal ingredients, and/or anti-inflammatories, analgesics, antioxidants, vitamins, etc. Specific exemplary active ingredients include honey, bee propolis extract, tocotrienols, vitamin E, ascorbic acid, superoxide dismutase, catalase, astaxanthin, lycopene, resveratrol, and allantoin.

While the amount of the additional active ingredient may vary from application to application, in accordance with various illustrative examples of the present invention, the additional active ingredient is present in an amount of about 0.01 to about 1.2 weight percent of the composition.

The following non-limiting example illustrates an exemplary composition in accordance with one embodiment of the invention. This example is merely illustrative, and it is not intended that the invention be limited to this example. Compositions in accordance with the present invention may include the ingredients listed below as well as additional and/or alternative inert materials, active ingredients, preservatives, and other constituents typically found in compositions for treating similar conditions. In the cases where exemplary inert, additional active materials, and/or preservatives are listed, these ingredients are merely exemplary, and it is understood that other similar ingredients may be substituted for the materials listed in.the examples below.

The exemplary composition listed below may be used for a variety of purposes. For example, the compositions can be used to treat an HSV infection. As noted above, such composition may alternatively be used to treat other forms of infections.

### EXAMPLE 1

| | | | Exemplary |
|---|---|---|---|
| | | Exemplary | Composition |
| | | Range (weight | (weight |
| Ingredient | Function | percent) | percent) |
| Syncrowax HGL-C | occlusive | 5-20 | 8.7 4036 |
| siliconized bee wax | binder/thickener | 5-10 | 3.5 |
| lanolin | emollient | 10-20 | 7.0 |
| Syncrowax HR-C | skin conditioner/ | 5-10 | 3.5 |
| (tribehenin) | occlusive | | |
| Syncrowax ERL-C | emollient | 5-10 | 2.6 |
| (C18-36 acid glycol | | | |
| ester) | | | |
| castor oil | emollient | 30-60 | 52 |
| jojoba oil | emollient | 20-40 | 18.3 |
| safflower oil | emollient | 2-4 | 1.7 |
| evening primrose oil | emollient | 2-4 | 1.9 |
| honey | thickener | 0.01-1 | 0.1 |
| bee propolis | antimicrobial | 0.1-2 | 0.1 |
| Peperina essential oil | antimicrobial | 0.1-2 | 0.7 |

The composition of Example 1 is formed by admixing 350 gram Syncrowax HGL-C (C18-36 acid triglyceride), 140 gm. siliconized bee wax, 280 gm. lanolin, 140 gm. Syncrowax HR-C (tribehenin), 105 gm. Syncrowax ERL-C (C18-36 acid glycol ester), 2107 gm. castor oil, 737 gm. jojoba oil, 70 gm. safflower oil 78 gm. evening primrose oil, 1.4 gm. honey, 1 gm. bee propolis 60% extract in propylene glycol, and 27 gm. Peperina essential oil.

Two humans applied the composition of Example 1 (in the form of a lip balm) to an infected area three times a day, starting at the beginning of a herpes labialis flare and reported that the duration of the flare was shorter than usual and that the pain and itch caused by the lesions had been alleviated.

In accordance with an additional embodiment of the invention, the composition as described above is used in connection with a tea-like bag with Peperina leaves, to be taken as an infusion. The tea-like bag may also be used topically to prevent and treat ocular herpes, or a suitable solution or gel could be used for this purpose.

Having described the invention with reference to particular compositions, it will be apparent to those of skill in the art that it is not intended that the invention be limited by such illustrative embodiments, and that modifications can be made without departing from the scope or spirit of the invention, as defined by the appended claims. It is intended that all modifications and variations be included within the scope of the invention. The claims are meant to cover the claimed components and steps in any sequence, unless the context specifically indicates the contrary.

We claim:

1. A method for the treatment and prevention of oral or perioral region, eyes, nongenital and genital skin or mucous membrane lesions caused by the human herpes simplex virus I, the method comprising the step of applying a composition containing Peperina essential oil and a carrier comprising a thickening agent to the affected area.

2. The method of claim 1, wherein the step of applying further comprises providing a carrier including resveratrol.

3. The method of claim 1, wherein the step of applying further comprises providing an ingredient selected from the group consisting of tocotrienols, vitamin E, ascorbic acid, superoxide dismutase, catalase, astaxanthin, lycopene.

4. A composition for treating a topical infection, the composition comprising:
a carrier including a thickening agent; and
a therapeutic amount of an herb extract from an herb of the family Lamiaceae.

5. The composition of claim 4, further comprising an emollient.

6. The composition of claim 5, wherein the emollient is selected from the group consisting of lanolin, acid glycerol ester, castor oil, jojoba oil, safflower oil, andevening primrose oil.

7. The composition of claim 4, further comprising an antimicrobial agent.

8. The composition of claim 7, wherein the antimicrobial agent is selected from the group consisting of bee propolis and tea tree oil.

9. The composition of claim 4, wherein the herb extract is Peperina essential oil.

10. The composition of claim 4, further comprising an occlusive agent.

11. A composition for the treatment of an infection, the composition comprising:
an active agent comprising a extract from an herb of the family Lamiaceae; and
a carrier configure to maintain the active agent in contact with a surface of a patient for an extended period of time.

12. The composition of claim 11, wherein the active agent comprises Peperina essential oil.

13. A composition for treatment of an infection, the composition comprising:
an active agent comprising a extract from an herb of the family Lamiaceae; and
an a carrier comprising a moisturizing agent

14. The composition of claim 13, wherein the active agent comprises Peperina essential oil.
